SEQUENCE LISTING (21 sequences)

SEQ ID NO: 1 - protein, 150 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGVATVAEKTKEQVTNVGGAVVTGVTAVAQKTVEGAGSIAAATGFVKKDQL

SEQ ID NO: 2 - protein, 750 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVS

SEQ ID NO: 3 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 96 aa, Homo sapiens
SDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGG

SEQ ID NO: 5 - protein, 96 aa, Mus musculus; identical to SEQ ID NO: 4

SEQ ID NO: 6 - protein, 92 aa, Homo sapiens
ADEKPKEGVKTENNDHINLKVAGQDGSVVQFKIKRHTPLSKLMKAYCERQGLSMRQIRFRFDGQPINETDTPAQLEMEDEDTIDVFQQQTGG

SEQ ID NO: 7 - protein, 91 aa, Homo sapiens
SEEKPKEGVKTENDHINLKVAGQDGSVVQFKIKRHTPLSKLMKAYCERQGLSMRQIRFRFDGQPINETDTPAQLEMEDEDTIDVFQQQTGG

SEQ ID NO: 8 - protein, 75 aa, Saccharomyces cerevisiae
LIKVKTLTGKEIELDIDPNDKVSRIKERVEEKEGIPPSQQRLIYAGKQMADDKNAESYHLEGGSVLHLVLALRGG

SEQ ID NO: 9 - protein, 210 aa, Homo sapiens
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 10 - protein, 128 aa, synthetic construct
  REGION 1..128: note = NbSyn2 nanobody
MDGQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSH

SEQ ID NO: 11 - protein, 122 aa, synthetic construct
  REGION 1..122: note = NbSyn87 nanobody
EVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSC

SEQ ID NO: 12 - protein, 14 aa, synthetic construct
  REGION 1..14: note = epitope of alpha synuclein recognized by NbSyn87
GAVVTGVTAVAQKT

SEQ ID NO: 13 - protein, 4 aa, synthetic construct
  REGION 1..4: note = epitope of alpha synuclein recognized by NbSyn2
SIAA

SEQ ID NO: 14 - protein, 150 aa, synthetic construct
  REGION 1..150: note = Alpha-Synuclein Ser87Cys
MDVFMKGLSKAKEGVVAAAEKTKQGVAEAAGKTKEGVLYVGSKTKEGVVHGVATVAEKTKEQVTNVGGAVVTGVTAVAQKTVEGAGCIAAATGFVKKDQLGVATVAEKTKEQVTNVGGAVVTGVTAVAQKTVEGAGSIAAATGFVKKDQL

SEQ ID NO: 15 - DNA, 7815 bp, synthetic construct
  misc_feature 1..7815: note = pSUMO1 plasmid
  source 1..7815: mol_type = other DNA
ttaatacgactcactataggggaattgtgagcggataacaattcccctgtagaaataattttgtttaactttaataaggagatataccatgggcagcagccatcaccatcatcaccacagccaggatctggttccgcgtatgtctgaccaggaggccaaaccttcaactgaggacttgggggataagaaggaaggtgaatatattaaactcaaagtcattggacaggatagcagtgagattcacttcaaagtgaaaatgacaacacatctcaagaaactcaaagaatcatactgtcaaagacagggtgttccaatgaattcactcaggtttctctttgagggtcagagaattgctgataatcatactccaaaagaactgggaatggaggaagaagatgtgattgaagtttatcaggaacaaacggggggttagggatccccgggaattcatcgtgactgactgacgctgcaggtcgacaagcttgcggccgcataatgcttaaccccttggggcctctaaacgggtcttgaggggttttttgcatgcatcgtattgtacacggccgcataatcgaaattaatacgactcactataggggaattgtgagcggataacaattccccatcttagtatattagttaagtataagaaggagatatacatatgtcggggatcgccctcagcagactcgcccaggagaggaaagcatggaggaaagaccacccatttggtttcgtggctgtcccaacaaaaaatcccgatggcacgatgaacctcatgaactgggagtgcgccattccgggaaagaaagggactccgtgggaaggaggcttgtttaaactacggatgcttttcaaagatgattatccatcttcgccaccaaaatgtaaattcgaaccaccattatttcacccgaatgtgtacccttcggggacagtgtgcctgtccatcttagaggaggacaaggactggaggccagccatcacaatcaaacagatcctattaggaatacaggaacttctaaatgaaccaaatatccaagacccagctcaagcagaggcctacacgatttactgccaaaacagagtggagtacgagaaaagggtccgagcacaagccaagaagtttgcgccctcataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacggtaccattaagtcgaacagaaagtaatcgtattgtacacggccgcataatcgaaattaatacgactcactataggggaattgtgagcggataacaattccccatcttagtatattagttaagtataagaaggagatatacatatggcagatctcaattcccgggtcgacatggcactgtcgcgggggctgccccgggagctggctgaggcggtggccgggggccgggtgctggtggtgggggcgggcggcatcggctgcgagctcctcaagaatctcgtgctcaccggtttctcccacatcgacctgattgatctggatactattgatgtaagcaacctcaacagacagtttttgtttcaaaagaaacatgttggaagatcaaaggcacaggttgccaaggaaagtgtactgcagttttacccgaaagctaatatcgttgcctaccatgacagcatcatgaaccctgactataatgtggaatttttccgacagtttatactggttatgaatgctttagataacagagctgcccgaaaccatgttaatagaatgtgcctggcagctgatgttcctcttattgaaagtggaacagctgggtatcttggacaagtaactactatcaaaaagggtgtgaccgagtgttatgagtgtcatcctaagccgacccagagaacctttcctggctgtacaattcgtaacacaccttcagaacctatacattgcatcgtttgggcaaagtacttgttcaaccagttgtttggggaagaagatgctgatcaagaagtatctcctgacagagctgaccctgaagctgcctgggaaccaacggaagccgaagccagagctagagcatctaatgaagatggtgacattaaacgtatttctactaaggaatgggctaaatcaactggatatgatccagttaaactttttaccaagctttttaaagatgacatcaggtatctgttgacaatggacaaactatggcggaaaaggaaacctccagttccgttggactgggctgaagtacaaagtcaaggagaagaaacgaatgcatcagatcaacagaatgaaccccagttaggcctgaaagaccagcaggttctagatgtaaagagctatgcacgtcttttttcaaagagcatcgagactttgagagttcatttagcagaaaagggggatggagctgagctcatatgggataaggatgacccatctgcaatggattttgtcacctctgctgcaaacctcaggatgcatattttcagtatgaatatgaagagtagatttgatatcaaatcaatggcagggaacattattcctgctattgctactactaatgcagtaattgctgggttgatagtattggaaggattgaagattttatcaggaaaaatagaccagtgcagaacaatttttttgaataaacaaccaaacccaagaaagaagcttcttgtgccttgtgcactggatcctcccaaccccaattgttatgtatgtgccagcaagccagaggtgactgtgcggctgaatgtccataaagtgactgttctcaccttacaagacaagatagtgaaagaaaaatttgctatggtagcaccagatgtccaaattgaagatgggaaaggaacaatcctaatatcttccgaagagggagagacggaagctaataatcacaagaagttgtcagaatttggaattagaaatggcagccggcttcaagcagatgacttcctccaggactatactttattgatcaacatccttcatagtgaagacctaggaaaggacgttgaatttgaagttgttggtgatgctccggaaaaagtggggcccaaacaagctgaagatgctgccaaaagcataaccaatggcagtgatgatggagctcagccctccacctccacagctcaagagcaagatgacgttctcatagttgattcggatgaagaagattcttcaaataatgccgacgtcagtgaagaagagagaagccgcaagaggaaattagatgagaaagagaatctcagtgcaaagaggtcacgtatagaacagaaggaagagcttgatgatgtcatagcattagattgagcggccgcattaaaaggaggaattaactatggtggagaaggaggaggctggcggcggcattagcgaggaggaggcggcacagtatgaccggcagatccgcctgtggggactggaggcccagaaacggctgcgggcctctcgggtgcttcttgtcggcttgaaaggacttggggctgaaattgccaagaatctcatcttggcaggagtgaaaggactgaccatgctggatcacgaacaggtaactccagaagatcccggagctcagttcttgattcgtactgggtctgttggccgaaatagggctgaagcctctttggagcgagctcagaatctcaaccccatggtggatgtgaaggtggacactgaggatatagagaagaaaccagagtcatttttcactcaattcgatgctgtgtgtctgacttgctgctccagggatgtcatagttaaagttgaccagatctgtcacaaaaatagcatcaagttctttacaggagatgtttttggctaccatggatacacatttgccaatctaggagagcatgagtttgtagaggagaaaactaaagttgccaaagttagccaaggagtagaagatgggcccgacaccaagagagcaaaacttgattcttctgagacaacgatggtcaaaaagaaggtggtcttctgccctgttaaagaagccctggaggtggactggagcagtgagaaagcaaaggctgctctgaagcgcacgacctccgactactttctccttcaagtgctcttaaagttccgtacagataaaggaagagatcccagttctgatacatatgaggaagattctgagttgttgctccagatacgaaatgatgtgcttgactcactgggtattagtcctgacctgcttcctgaggactttgtcaggtactgcttctccgagatggccccagtgtgtgcggtggttggagggattttggcacaggaaattgtgaaggccctgtctcagcgggaccctcctcacaacaacttcttcttcttcgatggcatgaaggggaatgggattgtggagtgccttggccccaagggctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaa

SEQ ID NO: 16 - DNA, 1200 bp, synthetic construct
  misc_feature 1..1200: note = His6-tag-hSUMO-NbSyn2
  source 1..1200: mol_type = other DNA
catcaccatcatcaccacagccaggatctggttccgcgtatgtctgaccaggaggccaaaccttcaactgaggacttgggggataagaaggaaggtgaatatattaaactcaaagtcattggacaggatagcagtgagattcacttcaaagtgaaaatgacaacacatctcaagaaactcaaagaatcatactgtcaaagacagggtgttccaatgaattcactcaggtttctctttgagggtcagagaattgctgataatcatactccaaaagaactgggaatggaggaagaagatgtgattgaagtttatcaggaacaaacggggggtatggatggccagctggttgaaagcggtggtggtagcgttcaggcaggcggtagcctgcgtctgagctgtgcagcaagcggtattgatagcagcagctattgtatgggttggtttcgtcagcgtccgggtaaagaacgtgaaggtgttgcacgtattaatggtctgggtggtgttaaaaccgcatatgcagatagcgttaaagatcgctttaccattagccgtgataatgccgaaaataccgtttacctgcagatgaatagcctgaaaccggaagataccgcaatctattattgtgcagccaaatttagtccgggttattgcggtggtagctggtcaaattttggttattggggtcagggcacccaggttaccgttagcagccatagggatccccgggaattcatcgtgactgactgacgctgcaggtcgacaagcttgcggccgcataatgcttaaccccttggggcctctaaacgggtcttgaggggttttttgcatgcatcgtattgtacacggccgcataatcgaaattaatacgactcactataggggaattgtgagcggataacaattccccatcttagtatattagttaagtataagaaggagatatacatatgtcggggatcgccctcagcagactcgcccaggagaggaaagcatggaggaaagaccacccatttggtttcgtggctgtcccaacaaaaaatcccgatggcacgatgaacctcatgaactgggagtgcgccattccgggaaagaaagggactccgtgggaaggaggcttgtttaaactacggatgcttttcaaagatgattatccatcttcgcaccaaaatgtaaattcgaaccaccattatttcacccgaa

SEQ ID NO: 17 - protein, 248 aa, synthetic construct
  REGION 1..248: note = His6-tag-hSUMO-NbSyn2
HHHHHHSQDLVPRMSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMDGQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSHRDPREFIVTD

SEQ ID NO: 18 - DNA, 1117 bp, synthetic construct
  misc_feature 1..1117: note = His-14-tag-scSUMO-Nbsyn87
  source 1..1117: mol_type = other DNA
catgagcaagcatcaccatcattcaggccatcaccataccggacaccaccatcattcaggcagtcatcaccataccggcggatcatccggaaataagttcaacaaggaacaacaaaacgccttttacgaaatcttgcatttgccgaatctgaacgaagaacaaaggaatggttttattcaatcattaaaagatgatccatcacaatcagctaatctacttgcagaagccaagaaattgaatgacgcccaggcacccaagggatcaaataagtttaataaagaacagcaaaacgctttttacgaaattctgcatctccctaatttgaatgaggaacaacgtaacggttttatacagtcattgaaggatgatccaagccagtccgctaacctattagctgaagctaagaaacttaacgacgcccaagctccaaagagctcaaccggatcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggcggaggcgggggtaccgaagttcagctgcaagaaagcggtggtggtagcgttcagaccggtggtagcctgcgtctgagctgtgttgcaagcggttatagtggttatatggcatggtttcgtcaggcaccgggtaaagaacgtgaaggtattgcagcaatttatcgcggtgataaaatcacctattatgcacatagcgtgcagggtcgttttaccattagccaggcaaatgcaaaaaataccgtttacctgctgatgaatagcctgaaaccggaagataccgcaatctattattgtgcagcacgtcgtgttgttgcagatagtccgctgctgagcaaaacctatgcatattggggtcagggcacccaggttaccgttagctgt

SEQ ID NO: 19 - protein, 372 aa, synthetic construct
  REGION 1..372: note = His-14-tag-scSUMO-Nbsyn87
MSKHHHHSGHHHTGHHHHSGSHHHTGGSSGNKFNKEQQNAFYEILHLPNLNEEQRNGFIQSLKDDPSQSANLLAEAKKLNDAQAPKGSNKFNKEQQNAFYEILHLPNLNEEQRNGFIQSLKDDPSQSANLLAEAKKLNDAQAPKSSTGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGGGGTEVQLQESGGGSVQTGGSLRLSCVASGYSGYMAWFRQAPGKEREGIAAIYRGDKITYYAHSVQGRFTISQANAKNTVYLLMNSLKPEDTAIYYCAARRVVADSPLLSKTYAYWGQGTQVTVSC

SEQ ID NO: 20 - DNA, 5039 bp, synthetic construct
  misc_feature 1..5039: note = K63 GGGG SynNb87 cys plasmid
  source 1..5039: mol_type = other DNA
ctcgagaaatcataaaaaatttatttgctttgtgagcggataacaattataatagattcaattgtgagcggataacaatttcacacagaattcattaaagaggagaaattaaccatgagcaagcatcaccatcattcaggccatcaccataccggacaccaccatcattcaggcagtcatcaccataccggcggatcatccggaaataagttcaacaaggaacaacaaaacgccttttacgaaatcttgcatttgccgaatctgaacgaagaacaaaggaatggttttattcaatcattaaaagatgatccatcacaatcagctaatctacttgcagaagccaagaaattgaatgacgcccaggcacccaagggatcaaataagtttaataaagaacagcaaaacgctttttacgaaattctgcatctccctaatttgaatgaggaacaacgtaacggttttatacagtcattgaaggatgatccaagccagtccgctaacctattagctgaagctaagaaacttaacgacgcccaagctccaaagagctcaaccggatcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggcggaggcgggggtaccgaagttcagctgcaagaaagcggtggtggtagcgttcagaccggtggtagcctgcgtctgagctgtgttgcaagcggttatagtggttatatggcatggtttcgtcaggcaccgggtaaagaacgtgaaggtattgcagcaatttatcgcggtgataaaatcacctattatgcacatagcgtgcagggtcgttttaccattagccaggcaaatgcaaaaaataccgtttacctgctgatgaatagcctgaaaccggaagataccgcaatctattattgtgcagcacgtcgtgttgttgcagatagtccgctgctgagcaaaacctatgcatattggggtcagggcacccaggttaccgttagctgttgagcggccgcacatcaccatcaccatcactaagcttaattagctgagcttggactcctgttgatagatccagtaatgacctcagaactccatctggatttgttcagaacgctcggttgccgccgggcgttttttattggtgagaatccaagctagccatgaaaataaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcttggttactcacgactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcacaaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggggccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtcttcac

SEQ ID NO: 21 - DNA, 8225 bp, synthetic construct
  misc_feature 1..8225: note = aSynS87C plasmid
  source 1..8225: mol_type = other DNA
gaatctcatcttggcaggagtgaaaggactgaccatgctggatcacgaacaggtaactccagaagatcccggagctcagttcttgattcgtactgggtctgttggccgaaatagggctgaagcctctttggagcgagctcagaatctcaaccccatggtggatgtgaaggtggacactgaggatatagagaagaaaccagagtcatttttcactcaattcgatgctgtgtgtctgacttgctgctccagggatgtcatagttaaagttgaccagatctgtcacaaaaatagcatcaagttctttacaggagatgtttttggctaccatggatacacatttgccaatctaggagagcatgagtttgtagaggagaaaactaaagttgccaaagttagccaaggagtagaagatgggcccgacaccaagagagcaaaacttgattcttctgagacaacgatggtcaaaaagaaggtggtcttctgccctgttaaagaagccctggaggtggactggagcagtgagaaagcaaaggctgctctgaagcgcacgacctccgactactttctccttcaagtgctcttaaagttccgtacagataaaggaagagatcccagttctgatacatatgaggaagattctgagttgttgctccagatacgaaatgatgtgcttgactcactgggtattagtcctgacctgcttcctgaggactttgtcaggtactgcttctccgagatggccccagtgtgtgcggtggttggagggattttggcacaggaaattgtgaaggccctgtctcagcgggaccctcctcacaacaacttcttcttcttcgatggcatgaaggggaatgggattgtggagtgccttggccccaagggctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggcagcagccatcaccatcatcaccacagccaggatctggttccgcgtatgtctgaccaggaggccaaaccttcaactgaggacttgggggataagaaggaaggtgaatatattaaactcaaagtcattggacaggatagcagtgagattcacttcaaagtgaaaatgacaacacatctcaagaaactcaaagaatcatactgtcaaagacagggtgttccaatgaattcactcaggtttctctttgagggtcagagaattgctgataatcatactccaaaagaactgggaatggaggaagaagatgtgattgaagtttatcaggaacaaacggggggtatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggtgcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaattcatcgtgactgactgacgctgcaggtcgacaagcttgcggccgcataatgcttaaccccttggggcctctaaacgggtcttgaggggttttttgcatgcatcgtattgtacacggccgcataatcgaaattaatacgactcactataggggaattgtgagcggataacaattccccatcttagtatattagttaagtataagaaggagatatacatatgtcggggatcgccctcagcagactcgcccaggagaggaaagcatggaggaaagaccacccatttggtttcgtggctgtcccaacaaaaaatcccgatggcacgatgaacctcatgaactgggagtgcgccattccgggaaagaaagggactccgtgggaaggaggcttgtttaaactacggatgcttttcaaagatgattatccatcttcgccaccaaaatgtaaattcgaaccaccattatttcacccgaatgtgtacccttcggggacagtgtgcctgtccatcttagaggaggacaaggactggaggccagccatcacaatcaaacagatcctattaggaatacaggaacttctaaatgaaccaaatatccaagacccagctcaagcagaggcctacacgatttactgccaaaacagagtggagtacgagaaaagggtccgagcacaagccaagaagtttgcgccctcataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacggtaccattaagtcgaacagaaagtaatcgtattgtacacggccgcataatcgaaattaatacgactcactataggggaattgtgagcggataacaattccccatcttagtatattagttaagtataagaaggagatatacatatggcagatctcaattcccgggtcgacatggcactgtcgcgggggctgccccgggagctggctgaggcggtggccgggggccgggtgctggtggtgggggcgggcggcatcggctgcgagctcctcaagaatctcgtgctcaccggtttctcccacatcgacctgattgatctggatactattgatgtaagcaacctcaacagacagtttttgtttcaaaagaaacatgttggaagatcaaaggcacaggttgccaaggaaagtgtactgcagttttacccgaaagctaatatcgttgcctaccatgacagcatcatgaaccctgactataatgtggaatttttccgacagtttatactggttatgaatgctttagataacagagctgcccgaaaccatgttaatagaatgtgcctggcagctgatgttcctcttattgaaagtggaacagctgggtatcttggacaagtaactactatcaaaaagggtgtgaccgagtgttatgagtgtcatcctaagccgacccagagaacctttcctggctgtacaattcgtaacacaccttcagaacctatacattgcatcgtttgggcaaagtacttgttcaaccagttgtttggggaagaagatgctgatcaagaagtatctcctgacagagctgaccctgaagctgcctgggaaccaacggaagccgaagccagagctagagcatctaatgaagatggtgacattaaacgtatttctactaaggaatgggctaaatcaactggatatgatccagttaaactttttaccaagctttttaaagatgacatcaggtatctgttgacaatggacaaactatggcggaaaaggaaacctccagttccgttggactgggctgaagtacaaagtcaaggagaagaaacgaatgcatcagatcaacagaatgaaccccagttaggcctgaaagaccagcaggttctagatgtaaagagctatgcacgtcttttttcaaagagcatcgagactttgagagttcatttagcagaaaagggggatggagctgagctcatatgggataaggatgacccatctgcaatggattttgtcacctctgctgcaaacctcaggatgcatattttcagtatgaatatgaagagtagatttgatatcaaatcaatggcagggaacattattcctgctattgctactactaatgcagtaattgctgggttgatagtattggaaggattgaagattttatcaggaaaaatagaccagtgcagaacaatttttttgaataaacaaccaaacccaagaaagaagcttcttgtgccttgtgcactggatcctcccaaccccaattgttatgtatgtgccagcaagccagaggtgactgtgcggctgaatgtccataaagtgactgttctcaccttacaagacaagatagtgaaagaaaaatttgctatggtagcaccagatgtccaaattgaagatgggaaaggaacaatcctaatatcttccgaagagggagagacggaagctaataatcacaagaagttgtcagaatttggaattagaaatggcagccggcttcaagcagatgacttcctccaggactatactttattgatcaacatccttcatagtgaagacctaggaaaggacgttgaatttgaagttgttggtgatgctccggaaaaagtggggcccaaacaagctgaagatgctgccaaaagcataaccaatggcagtgatgatggagctcagccctccacctccacagctcaagagcaagatgacgttctcatagttgattcggatgaagaagattcttcaaataatgccgacgtcagtgaagaagagagaagccgcaagaggaaattagatgagaaagagaatctcagtgcaaagaggtcacgtatagaacagaaggaagagcttgatgatgtcatagcattagattgagcggccgcattaaaaggaggaattaactatggtggagaaggaggaggctggcggcggcattagcgaggaggaggcggcacagtatgaccggcagatccgcctgtggggactggaggcccagaaacggctgcgggcctctcgggtgcttcttgtcggcttgaaaggacttggggctgaaattgccaa

(19) United States
(12) Patent Application Publication
(10) Pub. No.: US 2025/0199016 A1
(43) Pub. Date: Jun. 19, 2025

(54) METHOD FOR DETECTING MULTIMERIC, PREFERABLY DIMERIC PEPTIDES USING SINGLE-DOMAIN ANTIBODIES

(71) Applicant: Norelle Wildburger, Dallas, TX (US)

(72) Inventor: Norelle Wildburger, Dallas, TX (US)

(21) Appl. No.: 18/842,443

(22) PCT Filed: Feb. 27, 2023
(86) PCT No.: PCT/EP2023/054854

§ 371 Date: Aug. 29, 2024

(30) Foreign Application Priority Data
Mar. 3, 2022 (DE) 10 2022 105 061.0

(51) Int. Cl. G01N 33/68; C07K 14/435; C07K 16/18; G01N 33/543

(52) U.S. Cl. CPC G01N 33/6896; C07K 14/435; C07K 16/18; G01N 33/54353; G01N 33/6878; C07K 2317/622; G01N 2800/2835

(57) ABSTRACT

A method detects a multimeric, preferably dimeric, peptide containing a first and a second peptide monomer in a sample. The method includes providing a carrier material antibody conjugate comprising a carrier material to which a single domain carrier antibody specific for the first peptide monomer is bound; contacting the sample with the carrier material antibody conjugate, wherein a complex is formed between the carrier material antibody conjugate and the dimeric peptide, contacting the complex with a single domain detection antibody specific for the second peptide monomer comprising a detection marker, wherein the single domain detection antibody binds to the complex, wherein the single domain carrier antibody on the first peptide monomer and the single domain detection antibody on the second peptide monomer recognize the same epitope, and detecting the dimeric peptide using the detection marker. Such a method allows the targeted detection of multimeric or dimeric peptides in a sample.

In many neurodegenerative diseases, such as Alzheimer's or Parkinson's, protein aggregates can be detected in the brain or in neurons. Many of these protein aggregates are difficult to characterize wherein these often consist of poorly defined multimers, such as dimers or oligomers of often at least partially unfolded proteins, which makes detection for example by antibodies difficult. The detection of neurodegenerative diseases by antibodies is also made more difficult by heterophilic antibodies, which, as non-specific antibodies, cross-react with antigens of another species and can therefore trigger false positive test results. Immunoassays in which an analyte is detected by antibodies are often sensitive to cross-reacting substances that can give false test results. Another problem is that analytical antibodies are often very sensitive, so that incorrect handling of an immunoassay can lead to false test results.

One object of the present invention is to provide a method for the detection of multimers, preferably dimeric peptides, which allows a more reliable detection of multimeric peptides. A further object of the present invention is to provide a method for the detection of multimers, preferably dimeric peptides, in which the probability of cross-reacting heterophilic antibodies is reduced.

The object of the present invention is to provide a method for the detection of multimeric proteins which is improved with respect to the above-mentioned disadvantages. Another object of the invention is a method for detecting alpha-synuclein, which is also improved with respect to the above-mentioned disadvantages. Another object of the invention is a kit for carrying out the methods, expression constructs for expressing the specific antibodies, a positive control for carrying out the methods, and certain carrier material antibody conjugates which can be used in the methods.

Object of the present invention is a method for detecting a multimeric peptide, preferably a dimeric peptide, comprising a first and a second peptide monomer in a sample, comprising the method steps:
A) providing a carrier material antibody conjugate comprising a carrier material to which a single domain carrier antibody specific for the first peptide monomer is bound,
B) contacting the sample with the carrier material antibody conjugate, wherein a complex is formed between the carrier material antibody conjugate and the multimeric peptide,
C) contacting the complex with a single domain detection antibody specific for the second peptide monomer comprising a detection marker, wherein the single domain detection antibody binds to the complex, wherein the single domain carrier antibody on the first peptide monomer and the single domain detection antibody on the second peptide monomer recognize the same epitope, and
D) detecting the multimeric peptide using the detection marker.

In contrast to conventional antibodies, the method according to the invention uses single-domain antibodies. These are also referred to as “nano-antibodies” or “nanobodies” and are single, monomeric antibody fragments of a monomeric variable domain of an antibody. These single domain antibodies can be produced, for example, from the monomeric variable antigen-binding domains of heavy-chain antibodies produced by cartilaginous fish, such as sharks, or camels. The single domain antibodies are thus antigen-binding antibody fragments that are monomeric, in contrast to conventional antibodies, which are composed of two heavy chains and two light chains. These single domain antibodies are often considerably more stable than conventional antibodies, so that methods for detecting analytes based on single domain antibodies can be carried out more reliably. Furthermore, the occurrence of heterophilic, cross-reacting antibodies is significantly reduced when using single domain antibodies compared to conventional antibodies. Thus, detection methods based on single domain antibodies are significantly more reliable.

During method step B), the carrier material antibody conjugate recognizes the epitope on a first peptide monomer of the multimeric peptide via the single domain carrier antibody and binds the multimeric peptide to the carrier material via the first peptide monomer by complexation. The multimeric peptide immobilized on the carrier material can then be detected using a specific single domain detection antibody. A conjugate is formed out of the single domain detection antibody bound to the multimeric peptide and the multimeric peptide bound to the carrier material. The multimeric peptide is then detected via the detection marker, which is linked to the single domain detection antibody.

The single domain carrier antibody recognizes the same epitope on the first peptide monomer as the single domain detection antibody on the second peptide monomer.

Since the epitope is thus already occupied by the binding of the multimeric peptide to the single domain carrier antibody, a second peptide monomer within the multimeric peptide is required to provide the same epitope for binding to the single domain carrier antibody. The method according to the invention is thus capable of detecting dimeric peptides within multimeric protein agglomerates. The multimeric peptide can have any number of monomers, for example it can be a trimer or a tetramer with three or four monomeric peptides. Preference is given to the detection of a dimeric peptide in the method according to the invention. In particular, it is preferred that the method according to the invention can be used to detect a homo-dimer in which two identical peptide monomers form the dimer.

In method step B), the single domain carrier antibody binds to the first peptide monomer, wherein the epitope recognized by the single domain detection antibody on the first peptide monomer is blocked. The single domain detection antibody can therefore only bind the peptide on the second peptide monomer. Thus, the method according to the invention is particularly suitable for the detection of dimeric peptides also in protein agglomerates or protein oligomers.

Preferably, the method according to the invention can be used to detect dimers selected from: Beta amyloid peptide dimer, tau dimer, phosphorylated tau dimer or alpha-synuclein dimer. Preferably, the method is used for the detection of alpha-synuclein dimer.

In particular, the method according to the invention can be used to detect dimers in multimeric protein aggregates of beta amyloid, tau protein or alpha-synuclein.

Beta amyloid dimers, or aggregates of beta amyloid, respectively, are formed by cutting of the amyloid precursor protein by enzymes such as beta-secretase or gamma-secretase and are found in senile plaques in patients with Alzheimer's disease, for example. Deposits of the tau protein, which may contain the tau dimer, are for example also found in neurodegenerative diseases such as Alzheimer's or in chronic traumatic encephalopathy. These diseases are also known as tauopathies and are characterized, among other things, by the accumulation of aggregates of tau protein in the patient's brain.

Alpha-synuclein is a small soluble protein that among other things controls the release of dopamine in vertebrates. In neurodegenerative diseases such as Parkinson's disease or Lewvy body dementia, deposits of alpha-synuclein aggregates can be detected in the brain tissue of patients. The method according to the invention can thus also be used to detect synucleinopathies, which are characterized by the accumulation of aggregates of alpha-synuclein in the brain of patients.

The epitope recognized by the single domain carrier antibody and the single domain detection antibody on the first peptide monomer and on the second peptide monomer may, for example, be a protein sequence of a length between 10 to 40 amino acids, preferably between 15 to 30 amino acids, to which the single domain antibodies bind.

The multimeric peptide may in particular be a dimeric peptide. The dimeric peptide may comprise a monomer having a sequence identity of at least 80%, preferably 90%, more preferably at least 95%, more preferably identical to the amino acid sequence of SEQ. ID No. 1 (human alpha-synuclein), or SEQ. ID No. 2 (human tau protein) or SEQ. ID No. 3 (human beta-amyloid).

The percentage of identity of two nucleic acid sequences or two amino acid sequences can be determined using the algorithm of Thompson et al. (CLUSTALW, 1994 Nucleic Acid Research 22:4673-4, 680). A nucleotide sequence or an amino acid sequence may also be used as a so-called “query sequence” to perform a search of public nucleic acid or protein sequence databases to identify, for example, additional unknown homologous sequences which may also be used in embodiments of the present invention. Such searches can be performed using the algorithm of Karlin and Altschul (1999 Proceedings of the National Academy of Sciences U.S.A. 87:2, 264 to 2,268), modified as in Karlin and Altschul (1993 Proceedings of the National Academy of Sciences U.S.A. 90:5, 873 to 5,877). Such an algorithm is contained in the programs NBLAST and XBLAST by Altschul et al. (1999 Journal of Molecular Biology 215:403 to 410). The BLAST program can also be used (Altschul, S. F., Gish, W., Miller, W., Myers, E. W. & Lipman, D. J. (1990) J. Mol. Biol. 215:403-410). If there are gaps between two sequences, Gapped BLAST can be used, as described in Altschul et al. (1997 Nucleic Acid Research, 25:3, 389 to 3,402).

A person skilled in the art is aware that large plasmids can be produced not only by conventional cloning techniques, but also by techniques such as those described in US patents U.S. Pat. No. 6,472,184 B1 entitled “Method for Producing Nucleic Acid Polymers” and U.S. Pat. No. 5,750,380 entitled “DNA Polymerase Mediated Synthesis of Double Stranded Nucleic Acid Molecules”, which are hereby incorporated by reference in their entirety. These techniques allow the synthesis of large plasmids by solid phase synthesis without the need to amplify the plasmids in cell cultures.

In the method, a carrier material antibody conjugate can be used in which the single domain carrier antibody is bound to the carrier material via a peptide linker. The peptide linker comprises a length of at least 20 amino acids and comprises an alpha-helical region. Such a peptide linker is particularly suitable for binding the single domain carrier antibody to the carrier material without impairing its biological activity. Preferably, the peptide linker has a length between 30 to 130 amino acids, more preferably a length between 50 to 120 amino acids. Such lengths are particularly suitable for mobilizing the single domain carrier antibody to the carrier material without significantly impairing its biological activity. In the present invention, the peptide linker may, for example, comprise an amino acid sequence which comprises a sequence identity of at least 80%, preferably 90%, further preferably at least 95%, particularly preferably at least 98%, further particularly preferably at least 99% to the amino acid sequence of a SUMO protein, for example a human SUMO protein and/or a mouse SUMO protein and/or a yeast SUMO protein, such as the yeast SUMO protein ubiquitin-like protein SMT3, whose amino acid sequence is described under the identification number Q12306 (SMT3_YEAST) in the UniProt protein knowledgebase (UniProtKB). In particular, for example the proteins deposited in the UniProt protein knowledgebase under the following identification numbers are also “SUMO proteins” within the meaning of the present invention: P63165 (SUMO1_HUMAN), P61956 (SUMO2_HUMAN), P55854 (SUMO3_HUMAN), Q6EEV6 (SUMO4_HUMAN), G2XKQ0 (SUMO5_HUMAN), P63166 (SUMO1_MOUSE), P61957 (SUMO2_MOUSE), Q9Z172 (SUMO3_MOUSE) and 014399 (UBL1_SCHPO).

Preferably, the peptide linker comprises an alpha-helical region selected from the alpha-helical region of a SUMO peptide (small ubiquitin-like modifier peptide) or an FC region of an antibody. Preferably, the peptide linker comprises a sequence identity of at least 80%, preferably 90%, more preferably at least 95% to the amino acid sequence of the SEQ. ID No. 4 (human SUMO1), SEQ ID No. 5 (mouse SUMO), SEQ ID No. 6 (human SUMO2), SEQ ID No. 7 (human SUMO3), SEQ ID No. 8 (yeast SUMO), or SEQ ID No. 9 (FC region of a human antibody). These are fragments of the human SUMO sequences 1 to 3, a fragment of a mouse SUMO sequence and a fragment of a yeast SUMO sequence. The inventors of the present method have determined that these SUMO fragments and the FC region of a human antibody are particularly suitable for immobilizing single domain antibodies on a carrier material without loss of biological activity.

The peptide linker can be bound to the carrier material via a tag sequence. Such a tag sequence enables particularly reliable binding of the single domain carrier antibody to the carrier material via the peptide linker. The tag sequence is preferably a His tag sequence, which also preferably comprises a length of between three and 20 histidines. For example, a tag sequence with six histidines or a tag sequence with 14 histidines can be used for binding to the carrier material. Furthermore, it is also possible to use a GST tag that is a glutathione S-transferase. Such a GST tag can also bind well to carrier materials and thus enable the binding of the single domain carrier antibody to the carrier material via the peptide linker. The tag sequence with the peptide linker can be bound via the N-terminus or the C-terminus of the single domain carrier antibody. Preferably, the binding of the tag sequence and the peptide linker takes place at the N-terminus of the single-domain carrier antibody. Binding via the N-terminus of the single domain carrier antibody enables particularly good immobilization of the single domain antibody to the carrier material without loss of biological activity. The tag sequence can also determine the correct orientation of the single domain carrier antibody on the carrier material. In particular, this makes it possible to ensure that the single domain carrier antibody is immobilized on the carrier material on the one hand, but on the other hand is still able to bind the epitope of the first peptide monomer.

According to a further embodiment of a method according to the invention, the single domain carrier antibody and the single domain detection antibody can be the same single domain antibody. This makes it particularly easy to detect the same epitope on the first peptide monomer and the second peptide monomer of the multimeric peptide.

According to a further embodiment of a detection method according to the invention, the single domain carrier antibody and the single domain detection antibody are specific for alpha-synuclein. Preferably, the single domain carrier antibody and the single domain detection antibody have a sequence identity of at least 80%, preferably 90%, more preferably at least 95% to the amino acid sequence of SEQ. ID. No. 10 (single domain antibody NbSyn2), or to the amino acid sequence of SEQ. ID. No. 11 (single domain antibody NbSyn87), particularly preferably they are identical to the amino acid sequence of SEQ. ID. No. 10 (single domain antibody NbSyn2), or to the amino acid sequence of SEQ. ID. No. 11 (single domain antibody NbSyn87). The single domain antibody with the SEQ. ID. No. 10, the single domain antibody “NbSyn2” can bind in particular to the C-terminus of alpha-synuclein, which is particularly conserved between alpha-synuclein of different species. Thereby “NbSyn2” can recognize alpha-synuclein from various species, among others Homo Sapiens or the mouse (Mus musculus). In contrast, the single-domain antibody with the SEQ. ID. No. 11 is known as “Nbsyn87”. This single domain antibody binds to a less conserved region of human alpha-synuclein and is therefore suitable for the detection of primarily human alpha-synuclein.

The epitope on the first peptide monomer and the epitope on the second peptide monomer can be selected from an amino acid sequence with the SEQ. ID. No. 12 (amino acids 118-131 of alpha-synuclein recognized by NbSyn87) or SEQ. ID No. 13 (amino acids 137-140 of alpha-synuclein recognized by NbSyn2). The epitope with SEQ. ID No. 13 represents a highly conserved epitope of alpha-synuclein from various species and can therefore be used to detect alpha-synuclein from various sources. In contrast, the epitope with SEQ. ID. No. 12 is primarily suitable for the detection of human alpha-synuclein.

In method step A), a carrier material antibody conjugate can be provided, which carrier material comprises a plastic. Preferably, the plastic is selected from a group consisting of: Polyolefin, polystyrene, polyamide, polyurethane, polyvinyl chloride, phenolic polymers, nitrocellulose, latex, polysaccharide, composite material, ceramic, silicon dioxide, graphene and metal. Silicon dioxide can comprise silicon wafers, silicon nitride or glass as materials.

The carrier material preferably comprises a microtiter plate, microspheres, a microfluidic system (lab-on-a-chip system), or a microneedle patch that comprises one of the above-mentioned materials. Microtiter plates, microspheres or a microneedle patch are particularly suitable for immobilizing the single domain carrier antibody and to subsequently perform a binding with the single domain detection antibody in process step C) and the subsequent detection in process step D). The material of the microtiter plates, microspheres or the microneedle patch are preferably plastics selected from the group consisting of: Polyolefin, polystyrene, polyvinyl chloride, polyamide, particularly preferably polystyrene or polyvinyl chloride. Other preferred materials are graphene or silicon dioxide.

For example, a microtiter plate with wells isolated from each other can be used. In particular, a microtiter plate with 96 wells can be used. Such a microtiter plate can be used for an immunoassay according to a method according to the invention in which single domain antibodies are used.

In method step B) of a method according to the invention, the single domain carrier antibody of the carrier material antibody conjugate can bind to the dimeric peptide in a solution having a pH between 6.9 to 8, preferably a pH between 7.2 to 7.6 and an alkali metal salt concentration between 120 mM and 140 mM and a phosphate ion concentration between 10 mM and 15 mM.

In method step C) of a method according to the invention, the single domain detection antibody can bind to the dimeric peptide in a solution having a pH between 6.9 to 8, preferably a pH between 7.2 to 7.6 and an alkali metal salt concentration between 120 mM and 140 mM and a phosphate ion concentration between 10 mM and 15 mM.

These conditions correspond to physiological conditions and enable especially good binding of both the single-domain carrier antibody and the single-domain detection antibody to the epitopes of the dimeric peptide. A phosphate buffered saline (PBS) solution is particularly suitable for carrying out method steps B) and C). The solution contains 137 mM sodium chloride, 2.7 mM potassium chloride as alkali metal salts and 12 mM total phosphate (in the form of HPO₄²⁻ and H₂PO₄⁻). The pH value of the adjusted buffer solution is 7.4. The phosphate-buffered salt solution corresponds to the osmotic pressure of the human organism.

In one embodiment of a method according to the invention, a single domain detection antibody can be used in method step C), which comprises a detection marker selected from a group consisting of: Biotin, biotinylatable peptide sequence, preferably Avitag, chemiluminescent marker, and fluorescent marker.

Biotin as a detection marker binds strongly to streptavidin and avidin. These molecules can be coupled with enzymes such as horseradish oxidase, alkaline phosphatase or glucose peroxidase, which allow detection of the multimeric peptide, especially the dimeric peptide, by means of a color reaction. The alkaline phosphatase can, for example, convert as a dye substrate colorless p-nitrophenyl phosphate, which is converted into the weak yellow p-nitrophenol. In the case of peroxidase, o-phenylenediamine is used as dye substrate.

Instead of dye reactions, chemiluminescence reactions can also be used to detect the multimeric peptide. For example, a single-domain detection antibody coupled to a metal such as ruthenium can be used in the MSD reaction (Meso Scale Discovery). The microtiter plate comprises an electrode at the bottom of the wells. The binding of the single domain detection antibody coupled to ruthenium enables a redox reaction with the electrode in the wells, which produces light that can be detected by a CCD camera. Such an MSD detection method can be more sensitive than a dye reaction. It is also possible to use single domain detection antibodies that are bound to fluorescent molecules as detection markers, wherein detection using a laser is particularly sensitive, wherein the detection of individual molecules is also possible (Single Molecule Counting (SMC™) immunoassay technology).

Alternatively, a single domain detection antibody can be used that comprises an Avi tag at the C-terminus, a sequence that can be specifically labeled with biotin using the enzyme biotin ligase (BirA).

In a further embodiment of a method according to the invention, a positive control reaction can be carried out, wherein as dimeric peptide for the positive control reaction a homodimer is used whose monomer comprises a sequence identity of at least 80%, preferably 90%, more preferably at least 90% with the amino acid sequence of SEQ. ID. No. 14 (amino acid sequence of Ser87Cys), with the proviso that the amino acid cysteine at position 87 of SEQ. ID. No. 14 remains unchanged. Such a homodimer comprises a disulfide bridge due to the amino acid cysteine at position 87, which stabilizes the homodimer. Such a stabilized homodimer is particularly suitable as a positive control for a detection reaction according to the invention.

In method step B) of the method according to the invention, a sample of a brain fluid, spinal cord fluid, blood, serum, plasma, a saliva sample, a tissue biopsy, throat swab, nasal swab, urine, exosome diagnostics of a patient can be used. Such samples are particularly suitable for detecting a dimeric protein, especially alpha-synuclein.

The method according to the invention can be used in particular to determine whether a symptom-free patient is at risk of developing Parkinson's disease. Thereby, in particular alpha-synuclein dimer is detected in polymorphic peptide oligomers. The polymorphic peptide oligomers can comprise a variety of poorly characterized protein aggregates, such as dimers, trimers, tetramers or multimers.

It is also an object of the present invention to provide a method for detecting alpha-synuclein in a sample of a patient, comprising the method steps:
A) providing a carrier material antibody conjugate comprising a carrier material to which a single domain carrier antibody specific for alpha-synuclein is bound,
B) contacting the sample comprising alpha-synuclein with the carrier material antibody conjugate, wherein a complex is formed between the carrier material antibody conjugate and the alpha-synuclein,
C) contacting the complex with a single domain detection antibody specific for alpha-synuclein which comprises a detection marker, wherein the single domain detection antibody binds to the complex, and
D) detection of the alpha-synuclein using the detection marker.

Such a method enables due to the single domain carrier antibodies and the single domain detection antibodies a highly reliable detection of alpha-synuclein.

In such a method, especially monomers of alpha-synuclein can also be detected. Thereby, different single domain antibodies can be used as single domain carrier antibodies and as single domain detection antibodies that recognize different epitopes of alpha-synuclein.

Such a method allows monomers of alpha-synuclein to be detected particularly easily, as different epitopes are recognized on a monomeric alpha-synuclein. Such methods can be used in combination with methods for the detection of dimeric alpha-synuclein in alpha-synuclein agglomerates to determine a ratio of dimeric alpha-synuclein to monomeric alpha-synuclein. In neurodegenerative diseases, such as Alzheimer's, an increase in the ratio of dimeric alpha-synuclein to monomeric alpha-synuclein is to be expected.

According to a further embodiment of a method according to the invention, the method is used to detect Parkinson's in humans or to determine whether a symptom-free human patient is at risk of developing Parkinson's. As single domain carrier antibody a single domain antibody is used which comprises a sequence identity of at least 80%, preferably 90%, more preferably at least 95% to the amino acid sequence of SEQ. ID. No. 11 (NbSyn87). Particularly preferably, the single domain carrier antibody can be identical to the amino acid sequence of SEQ. ID. No. 11. The single domain antibody “NbSyn87” can, as already explained above, specifically detect a C-terminal end of human alpha-synuclein and is thus particularly suitable for the determination of Parkinson's disease in humans.

A further embodiment of a method according to the invention is used for the detection of alpha-synuclein in vertebrates selected from a group consisting of: Human, mouse, monkey, wherein as single domain carrier antibody a single domain antibody is used which comprises a sequence identity of at least 80%, preferably 90%, further preferably at least 95% to the amino acid sequence of SEQ. ID. No. 10 (NbSyn2). Particularly preferably, the single domain carrier antibody can be identical to the amino acid sequence of SEQ. ID. No. 10. As described above, the antibody “NbSyn2” can detect alpha-synuclein from various species, comprising human, mouse and monkey.

It is also an object of the present invention to provide a kit for carrying out a method as described above, wherein a multimeric peptide comprising a first and a second peptide monomer can be detected within a protein agglomerate in a sample. The kit comprises the following components:
a carrier material antibody conjugate comprising a carrier material to which a single domain carrier antibody specific for the first peptide monomer is bound,
a single domain detection antibody specific for the second peptide monomer comprising a detection marker,
reagents for detecting the dimeric peptide using the detection marker.

Such a kit can be used for the detection of multimeric peptides containing a first and a second peptide monomer. In particular, the kit is suitable for the detection of dimeric peptides containing a first and a second peptide monomer. Thereby, the kit can contain the above-mentioned reagents, in particular the carrier material antibody conjugates, the single domain detection antibodies with the detection marker and suitable detection reagents as described above.

The kit can further contain instructions for carrying out the methods described herein. In particular, the instructions can comprise step-by-step instructions for carrying out a method for detecting a multimeric peptide with the method steps A) to D) as described above.

In the kit, the single domain detection antibody and the single domain carrier antibody can be present in storage stable form, for example in lyophilized form. Furthermore, buffer solutions for carrying out the procedure, for example a PBS solution, can also be present in the kit. A washing solution for washing off unbound sample components can also be present, which enables the removal of unbound components of a sample after the complex between the carrier material antibody conjugate and the multimeric, preferably dimeric peptide has been formed. Likewise, the carrier material suitable for binding the single domain carrier antibody can already be present in the kit. In particular, this can be microtiter plates, microspheres or a microneedle patch.

The single domain carrier antibody and/or the single domain detection antibody of the kit can comprise a sequence identity of at least 80%, preferably 90%, more preferably at least 95% to the amino acid sequence of SEQ. ID. No. 10 (NbSyn2) or the amino acid sequence of SEQ. ID. No. 11 (NbSyn87). Particularly preferably, the single domain carrier antibody and/or the single domain detection antibody can be identical to the amino acid sequences of SEQ. ID. No. 11 or the amino acid sequence of SEQ. ID. No. 10.

The kit can further serve to detect an alpha-synuclein dimer and comprise as a standard for a positive control reaction a homodimer of alpha-synuclein, wherein the monomer comprises a sequence identity of at least 80%, preferably 90%, more preferably at least 90% with the amino acid sequence of SEQ. ID. No. 14 (alpha-synuclein Ser87Cys), with the proviso that the amino acid cysteine at position 87 of SEQ. ID. No. 14 remains unchanged. Such a homodimer comprises a disulfide bridge due to the amino acid cysteine at position 87, which stabilizes the homodimer. This protein is therefore particularly suitable as a standard.

Alternatively or additionally, the kit can also be used to detect an alpha-synuclein in a patient's sample, in which case monomeric alpha-synuclein can preferably be used as a standard in the kit.

In particular, the kit can be suitable for detecting alpha-synuclein in a sample, so that it further comprises:
a carrier material antibody conjugate comprising a carrier material to which a single domain carrier antibody specific for alpha-synuclein is bound,
a single domain detection antibody specific for alpha-synuclein which comprises a detection marker, and
reagents for detecting alpha-synuclein using the detection marker.

The kit can further also contain instructions for carrying out a method for detecting alpha-synuclein in a sample from a patient. This applies in particular to method steps A) to D), as described above for the detection of alpha-synuclein.

The kit can also be used to detect alpha-synuclein, wherein the single domain carrier antibody and the single domain detection antibody are different single domain antibodies that recognize different epitopes of alpha-synuclein. With the aid of such a kit, for example, a single domain carrier antibody immobilized on a carrier material can recognize a first epitope of alpha-synuclein in method step B), wherein in subsequent method step C) the single domain detection antibody then recognizes a second epitope of alpha-synuclein on the bound alpha-synuclein, which is different from the first epitope. Such a kit can thus detect alpha-synuclein in general, regardless of its aggregation state. The alpha-synuclein can thereby be present as monomer, dimer or multimer.

Thereby, in particular one of the single domain antibodies can comprise a sequence identity of at least 80%, preferably 90%, more preferably at least 95% to the amino acid sequence of SEQ. ID. No. 10. Preferably, one of the single domain antibodies can be identical to the amino acid sequence of SEQ. ID. No. 10. In this case, the other single domain antibody can comprise a sequence identity of at least 80%, preferably 90%, more preferably at least 95% to the amino acid sequence of SEQ. ID. No. 11. Preferably, the other single domain antibody can be identical to the amino acid sequence of SEQ. ID. No. 11.

For the detection of human alpha-synuclein, a single domain carrier antibody is preferably used which comprises a sequence identity of at least 80%, preferably 90%, more preferably at least 95% to the amino acid sequence of SEQ. ID. No. 11. Preferably, the single domain carrier antibody is identical to the amino acid sequence of SEQ. ID. No. 11. The single domain detection antibody used is then a single domain antibody which preferably comprises a sequence identity of at least 80%, preferably 90%, more preferably at least 95% to the amino acid sequence of SEQ. ID. No. 10. Preferably, the single domain detection antibody can be identical to the amino acid sequence of SEQ. ID. No. 10.

As explained above, the single domain antibody “NbSyn87” recognizes a region of human alpha-synuclein that is less conserved between different alpha-synuclein proteins and is therefore specific for human alpha-synuclein. In contrast, the single domain antibody “NbSyn2” recognizes a region conserved between different alpha-synuclein proteins from different species and thus allows the recognition of alpha-synuclein proteins from different species, for example Homo sapiens or the mouse (Mus musculus).

“NbSyn87” as single domain carrier antibody thus only allows the binding of human alpha-synuclein, which can be bound by the single-domain detection antibody ‘NbSyn2’ in the subsequent process step C) and detected in process step D), so that such a kit allows a detection specific for human alpha-synuclein.

For the detection of alpha-synuclein of different species, preferably a single domain carrier antibody is used, which comprises a sequence identity of at least 80%, preferably 90%, more preferably at least 95% to the amino acid sequence of SEQ. ID. No. 10 (NbSyn2). Preferably, the single domain carrier antibody is identical to the amino acid sequence of SEQ. ID. No. 10. The same single domain antibody is then used as the single domain detection antibody, which preferably comprises a sequence identity of at least 80%, preferably 90%, more preferably at least 95% to the amino acid sequence of SEQ. ID. No. 10 (NbSyn2). Preferably, one of the single domain detection antibodies can be identical to the amino acid sequence of SEQ. ID. No. 10.

The single domain carrier antibody comprising a sequence identity of at least 80%, preferably 90%, more preferably at least 95% to the amino acid sequence of SEQ. ID. No. 10 can be used to detect alpha-synuclein from species such as Homo sapiens (human), Mus musculus (mouse), Rattus norvegicus (rat), Gorilla gorilla gorilla (western lowland gorilla). Macaca fascicularis (Javan monkey), Pan paniscus (bonobo). Pan troglodytes (chimpanzee), Bos taurus (bovine), Serinus canaria (Canary chaffinch), Macaca mulatta (rhesus macaque), Sus scrofa (pig), Pongo abelii (Sumatran orangutan) (Pongo pygmaeus abelii). Ateles geoffroyi (Geoffroy's spider monkey). Erythrocebus patas (red guenons) (Cercopithecus patas), Lagothrix lagotricha (brown woolly monkey) (Humboldt's woolly monkey), Saguinus labiatus (red-bellied tamarin) and Xenopus laevis (African clawed frog).

A kit with such a single domain carrier antibody and single domain detection antibody allows the binding of alpha-synuclein of different species to the single domain carrier antibody and thus allows the detection of different alpha-synuclein proteins.

An object of the present invention is also an alpha-synuclein homodimer, wherein the monomers have a sequence identity of at least 95%, further preferably identical to the amino acid sequence with the amino acid sequence of SEQ. ID. No. 14, with the proviso that the amino acid cysteine at position 87 of SEQ. ID. No. 14 remains unchanged.

Such an alpha-synuclein homodimer is especially stable due to the disulfide bridge between the cysteines of two monomers and is therefore particularly suitable as a positive control for the methods according to the invention.

An object of the present invention is also an expression construct comprising a gene which encodes an alpha-synuclein polypeptide, wherein the alpha-synuclein polypeptide has a sequence identity of at least 95%, further preferably identical to the amino acid sequence with the amino acid sequence of SEQ. ID. No. 14, with the proviso that the amino acid cysteine at position 87 of SEQ. ID. No. 14 remains unchanged. The expression construct can comprise, for example, the expression plasmid “pSumo1” of the non-profit plasmid archive “Addgene”, into which the gene which encodes the above-mentioned alpha-synuclein polypeptide has been inserted. The nucleotide sequence of pSUMO1 is the nucleotide sequence with SEQ ID No. 15.

The expression construct can then be integrated into expression-competent host cells, for example E. coli cells such as Rosetta™ 2 (pLysS) from Novage.

Another object of the present invention is an expression construct comprising a gene which encodes a single domain antibody specific for a peptide monomer with a peptide linker attached to the single domain antibody, wherein the peptide linker comprises a length of at least 20 amino acids and comprises an alpha-helical region.

The single domain antibody encoded by the expression construct can be specific for alpha-synuclein, the tau protein or for beta amyloid. The specific single domain antibody encoded by the gene can be specific for a peptide monomer comprising a sequence identity of at least 80%, preferably 90%, more preferably 95% to an amino acid sequence of the amino acid sequence of SEQ. ID No. 1 (alpha-synuclein). The single domain antibody can also be specific for beta amyloid. Such single domain antibodies are described in the publication Drews, A. et al. Sci. Rep. 6, 31910. The single domain antibody can also be specific for the tau protein. Such single domain antibodies are described in the publication Li et al. Journal of Controlled Release, 243, 2016, pages 1-10.

The single domain antibody encoded by the expression construct can comprise a sequence identity of at least 80%, preferably 90%, more preferably at least 95% to the amino acid sequence of SEQ. ID. No. 10 (NbSyn2) or the SEQ. ID. No. 11 (NbSyn87).

Particularly preferably, the single domain antibody can be identical to the amino acid sequence of SEQ. ID. No. 10 (NbSyn2) or the SEQ. ID. No. 11 (NbSyn87).

The expression construct can encode a peptide linker which comprises an alpha-helical region which is selected from the alpha-helical region of a SUMO peptide (small ubiquitin modifier peptide) or an FC region of an antibody. Preferably, the peptide linker comprises a sequence identity of at least 80%, preferably 90%, more preferably at least 95% to the amino acid sequence of the SEQ. ID No.4, 5, 6, 7, 8, or 9.

In particular, the expression construct can comprise an expression plasmid containing a gene which encodes the single domain antibody. In particular, the plasmid “pSUMO1” can be considered as an expression plasmid (SEQ. ID. No. 15), which allows the expression of the single domain antibody with a 6 His-SUMO tag at the N-terminus of the single domain antibody.

It is also an object of the invention to provide a carrier material antibody conjugate comprising a carrier material to which a single-domain carrier antibody is bound, wherein the single domain carrier antibody is bound to the carrier material via a peptide linker. Thereby, the peptide linker comprises a length of at least 20 amino acids and comprises an alpha-helical region. The peptide linker preferably has a length of between 30 and 130 amino acids, more preferably between 50 and 120 amino acids.

The peptide linker in the carrier material antibody conjugate can comprise a sequence identity of at least 80%, preferably 90%, more preferably at least 95% to the amino acid sequence of SEQ. ID No. 4, 5, 6, 7, 8, or 9.

In the carrier material antibody conjugate, the single domain carrier antibody can comprise a sequence identity of at least 80%, preferably 90%, more preferably at least 95% to the amino acid sequence of SEQ. ID. No. 10 or the SEQ. ID. No. 11. Preferably, the single domain carrier antibody is identical to the amino acid sequence of SEQ. ID. No. 10 or the SEQ. ID. No. 11.

The single domain carrier antibody can be bound to the carrier material via a tag, preferably via a His tag or a GST tag. Preferably, the single domain carrier antibody comprises a His tag.

The carrier material of the carrier material antibody conjugate can preferably be a microtiter plate, microspheres or a microneedle patch. Due to the peptide linker and the tag, the single domain carrier antibody can be immobilized to the carrier material in a correct orientation that allows binding to the multimeric peptide, in particular to the dimeric alpha-synuclein.

An object of the present invention is also a single domain antibody specific for a peptide monomer with a peptide linker bound to the single domain antibody, wherein the peptide linker comprises a length of at least 20 amino acids and comprises an alpha-helical region.

The single domain antibody can have a sequence identity of at least 80%, preferably 90%, more preferably at least 95%, more preferably identical to that of the amino acid sequence of SEQ. ID No. 11. The single domain antibody can have a sequence identity of at least 80%, preferably 90%, more preferably at least 95%, more preferably identical to the amino acid sequence of SEQ. ID No. 10.

The peptide linker in the single domain antibody can comprise a sequence identity of at least 80%, preferably 90%, more preferably at least 95% to the amino acid sequence of SEQ. ID No. 4, 5, 6, 7, 8, or No. 9.

The explanations and disclosures relating to an object according to the invention also apply mutatis mutandis to all other objects according to the invention, provided that they do not contradict the specific explanations and disclosures of the other objects according to the invention. For example, the explanations and disclosures relating to the method according to the invention also apply mutatis mutandis to the expression construct according to the invention, the carrier material antibody conjugate according to the invention, the kit according to the invention, the alpha-synuclein homodimer according to the invention, the peptide monomer according to the invention and the single domain antibody according to the invention, and vice versa, provided that these do not contradict the specific explanations and disclosures made in relation to the respective objects according to the invention. For example, the specific embodiments for the peptide linker disclosed in connection with the method according to the invention also apply with respect to the other objects according to the invention, provided that these explanations and disclosures do not contradict the specific explanations and disclosures made with respect to these objects according to the invention.

Further features and advantages of the invention are evident from the following special description and the drawings.

Identical reference signs in the figures indicate identical or analogous elements.

Of course, the embodiments discussed in the specific description and shown in the figures are only illustrative examples of the present invention. In the light of the present disclosure, the skilled person is provided with a wide range of possible variations.

It shows:

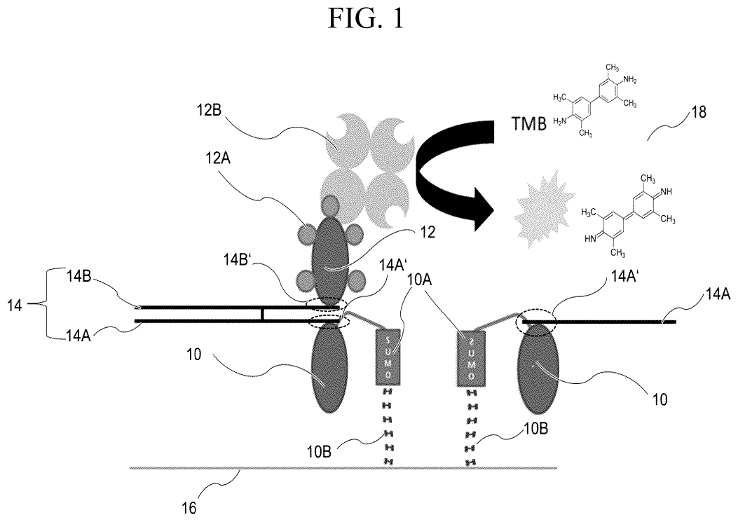

FIG. 1 is a schematic representation of a method for detecting a multimeric peptide, in particular a dimeric peptide using specific single domain antibodies;

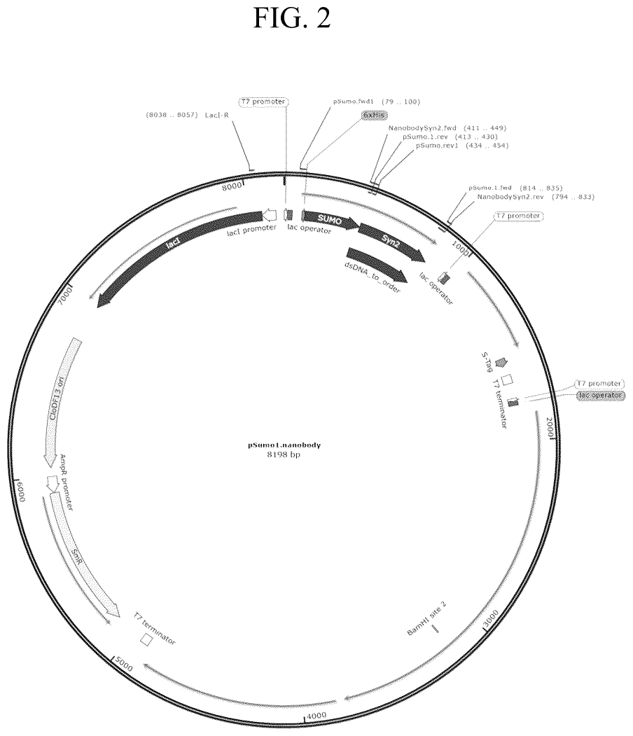

FIG. 2 a plasmid map of a plasmid comprising a sequence which encodes the single domain carrier antibody “NbSyn2” which is attached to a peptide linker which comprises a human sumo sequence;

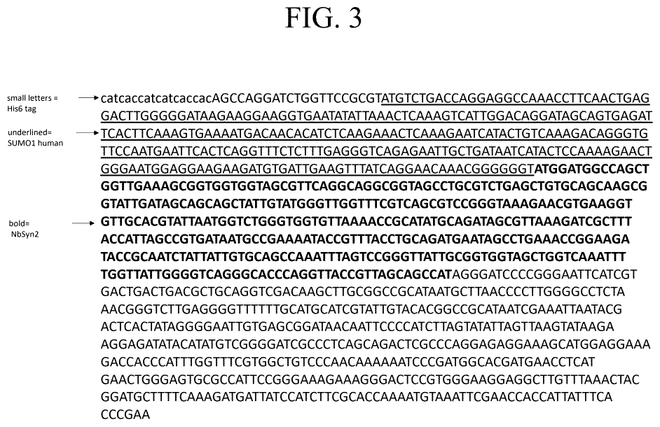

FIG. 3 the nucleic acid sequence of the single domain carrier antibody “NbSyn2”, which has a human sumo sequence and a His 6 tag at the N-terminus (SEQ ID. No. 16);

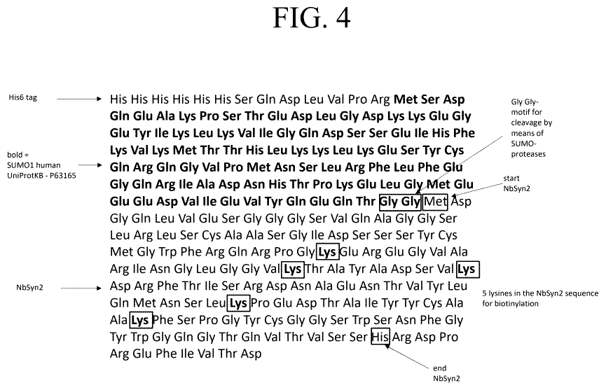

FIG. 4 the amino acid sequence of the single domain carrier antibody “NbSyn2” whose nucleic acid sequence is shown in FIG. 3 (SEQ ID. No. 17);

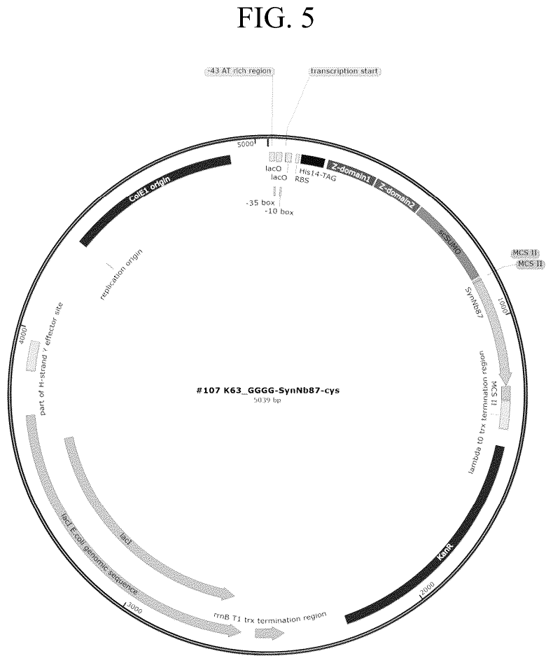

FIG. 5 a plasmid map of a plasmid which comprises a sequence which encodes the single domain carrier antibody “NbSyn87” which is attached to a peptide linker which comprises a yeast sumo sequence;

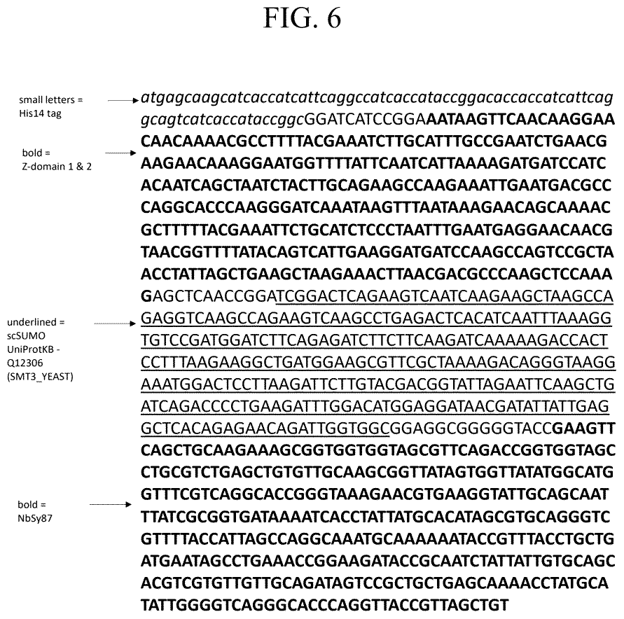

FIG. 6 a nucleic acid sequence of the single domain carrier antibody “NbSyn87” which comprises a yeast sumo sequence at the N-terminus as well as a Z-domain and a His 14-tag (SEQ ID. No. 18);

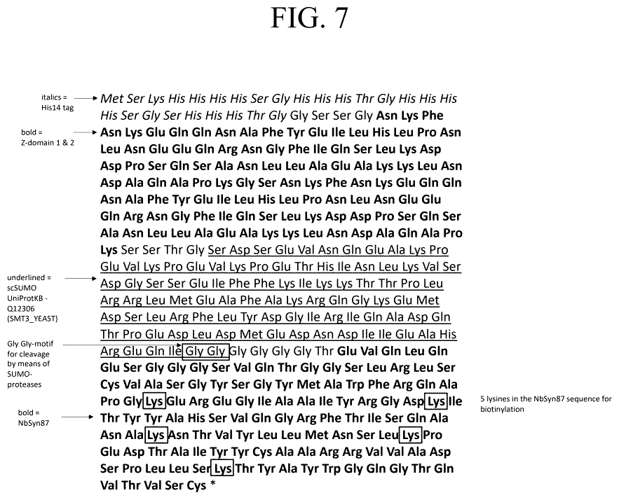

FIG. 7 shows the corresponding amino acid sequence of the single domain carrier antibody “NbSyn87” whose nucleic acid sequence is shown in FIG. 6 (SEQ ID. No. 19);

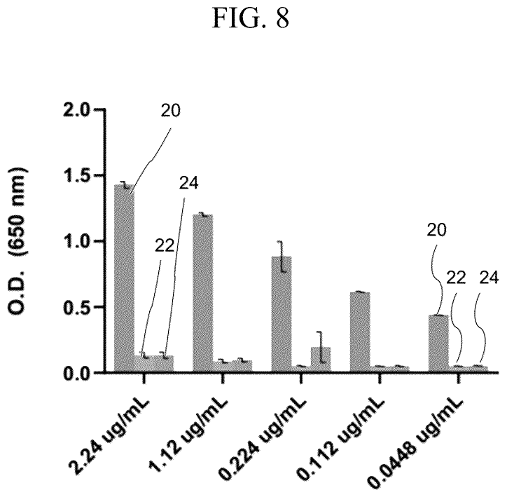

FIG. 8 the luminescence at a wavelength of 650 nm as a function of different concentrations of the sample of a brain lysate at a concentration of a single domain carrier antibody of 0.1 μg/ml;

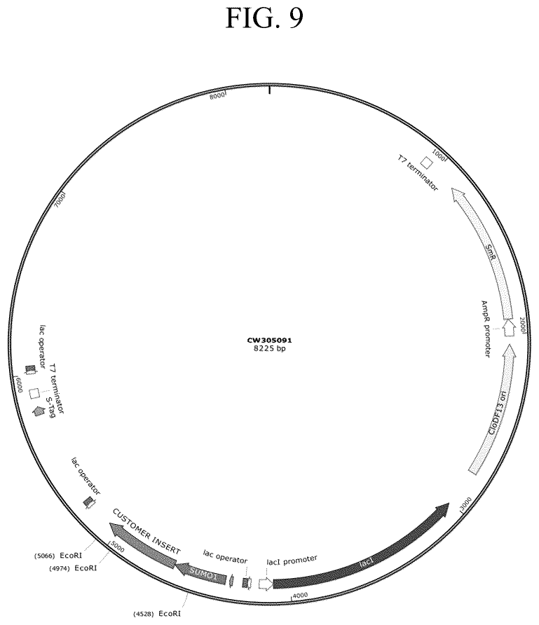

FIG. 9 a plasmid map of the plasmid “aSynS87C”, whose nucleotide sequence is described in SEQ ID. No. 21.

### PREFERRED EMBODIMENTS

FIG. 1 shows a schematic representation of a method according to the invention for detecting a multimeric peptide, in particular a dimeric peptide, using single domain antibodies. A single domain carrier antibody 10 is immobilized on a carrier material 16 by using a His tag 10B via a peptide linker 10A. This single domain carrier antibody 10 recognizes an epitope 14A′ on a first peptide monomer of the multimeric peptide. The multimeric peptide can thereby comprise a dimeric peptide 14 that contains a first peptide monomer 14A and a second peptide monomer 14B. The first peptide monomer 14A and the second peptide monomer 14B can in particular be identical, so that the dimeric peptide 14 is a homodimer. A single domain detection antibody 12 recognizes the epitope 14B′ on the second peptide monomer 14B. The epitope 14B′ of the second peptide monomer 14B is the same epitope as the epitope 14A′ of the first peptide monomer 14A. The single domain detection antibody 12 can thus only bind to the dimeric peptide 14 if the dimeric peptide comprises two identical epitopes 14A′ and 14B′. The single-domain detection antibody 12 comprises a detection marker 12A, which can be bound by a reporter enzyme 12B. The reporter enzyme then enables the detection of the complex between the carrier material antibody conjugate and the dimeric peptide via a color reaction 18. The left side of the schematic representation of FIG. 1 shows the detection of the dimeric peptide 14, while the right side of the schematic representation of FIG. 1 shows how the carrier material antibody conjugate binds a first peptide monomer 14A. Since this first peptide monomer 14A has only one epitope 14A′, but no further epitope 14B′, the single domain detection antibody cannot bind to the peptide monomer 14A, since its epitope is already bound and thus blocked by the single domain detection antibody 10. Therefore, no detection of the peptide monomer can take place if only peptide monomers are present in the sample to be analyzed. The method according to the invention is thus capable of detecting dimeric peptides even within multimeric protein aggregates without detecting the corresponding monomeric peptides.

FIG. 2 shows a plasmid map of the pSumo1 plasmid, which comprises a gene sequence that codes for the single-domain antibody “NbSyn2”, named “Syn2”. The plasmid map shows that the sumo sequence is present as a peptide linker at the N-terminus of the single domain antibody. A 6His tag is located at the N-terminus of the sumo sequence, which is used to bind the single domain antibody “Syn2” via the peptide linker to a carrier material, for example an ELISA plate. This plasmid can be used to express the single domain carrier antibody “Syn2” in, for example, E. coli. The nucleic acid sequence of the pSumo1 plasmid is described in SEQ ID. No. 15.

FIG. 3 shows the nucleic acid sequence of the single domain carrier antibody “NbSyn2”, wherein a human Sumo peptide sequence is present at its N-terminus as a peptide linker (shown underlined in FIG. 3). In this case, the human SUMO peptide sequence is a peptide sequence of the human SUMO1 protein (small ubiquitin-related modifier 1), which is listed, for example, in the UniProt protein knowledgebase (UniProtKB) under the identification number P63165. A 6His tag is present on this peptide linker (His tag shown in lower cases). At the C-terminus of the single domain carrier antibody, whose nucleic acid sequence is shown underlined, there is an additional sequence that was introduced into the single domain carrier antibody “NbSyn2” by cloning, but which has no effect on its activity.

FIG. 4 shows the amino acid sequence of the single domain carrier antibody “NbSyn2”, whose nucleic acid sequence is shown in FIG. 3. The amino acid sequence of the peptide linker Sumo is shown in bold. The amino acid sequence of the single-domain carrier antibody “NbSyn2” comprises 5 lysines, which can be used as detection markers for biotinylation. The biotins can be recognized by streptavidin-labeled reporter enzymes and used to detect the multimeric peptide by means of a color reaction. FIG. 4 also shows 10 amino acids of the additional sequence at the C-terminus of the single domain carrier antibody. For reasons of clarity, this sequence is shown in abbreviated form in FIG. 4.

FIG. 5 shows the plasmid map of the plasmid “K63 GGGG SynNb87 cys”, which comprises a section that encodes the single domain antibody “NbSyn87”. At the 5′ end, this section is linked to a gene section that encodes part of a yeast Sumo peptide sequence. In this case, the yeast SUMO peptide sequence (scSUMO) is a peptide sequence of the yeast SUMO protein ubiquitin-like protein SMT3, which is listed, for example, in the UniProt protein knowledgebase (UniProtKB) under the identification number Q12306. Z domains 1 and 2 are also present, as well as a 14 His tag for binding the single domain carrier antibody to a carrier material. This plasmid can also be used to express the single domain carrier antibody “NbSyn87” in E. coli. The nucleic acid sequence of the plasmid “K63 GGGG SynNb87 cys” is described in SEQ ID. No. 20.

FIG. 6 shows the nucleic acid sequence of the single domain carrier antibody “NbSyn87” (bold sequence “NbSyn87”) at whose 5′-end the SMT3 sumo-peptide sequence is present (underlined sequence), followed by the Z-domains 1 and 2 (bold sequence). Furthermore, a 14His tag is present for binding to the carrier material (shown in lower case in FIG. 6).

FIG. 7 shows the amino acid sequence of the nucleic acid sequence shown in FIG. 6. The bolded amino acid sequence of “NbSyn87” contains 5 lysines for biotinylation as detection markers. The SMT3 sumo-peptide sequence is underlined and the 14His tag is shown in italics.

### Introduction of a Point Mutation Ser87Cys in Alpha-Synuclein

The inventors have introduced a point mutation into the human alpha-synuclein so that pure homo-dimers, rather than a mixture of higher order oligomers, can be produced under redox conditions. Alpha-synuclein has 4 serines: serine 9, 42, 87 and 129. Serine 129 was excluded as a possible mutation site because it has an important function as a phosphorylation site in vivo. Serine 9 and 42 are both located in the N-terminal region of alpha-synuclein and would therefore most likely not form stable synthetic homodimers.

Therefore, the inventors introduced a point mutation of serine87cysteine. Measuring alpha-synuclein as units of “dimer equivalents” using these stoichiometric, well-characterized aSynSer87Cys dimers as an assay standard will allow for reproducible measurement.

The plasmid “aSynS87C” was used to express the point mutation of alpha-synuclein in E. coli. FIG. 9 shows the plasmid map of the plasmid “aSynS87C”, which was obtained by cloning an insert encoding the point mutant of alpha-synuclein S87C into the vector pSUMO1 in the region described as “customer insert” in the plasmid map. A His tag and the SUMO1 sequence were attached to the N-terminus of the point mutant.

### Production of Synthetic Dimer Standards

Purified recombinant monomeric aSyn with the point mutation S87C was dimerized according to a protocol adapted from O'Nuallian et al, 2010 (O'Nuallain et al, J Neurosci 30 (43), 2010, 14411-14419). In brief, 40 uM aSynS87C was diluted 1:1 with 20 mM ammonium bicarbonate (pH 8.2) and incubated for five days at 25° C. and 300 rpm in a thermomixer. Larger aggregates were dissolved by lyophilizing the dimer reaction and resuspending in 50 mM Tris-HCl with 5 M guanidine (pH 8.0) overnight. The next day, the samples were subjected to size exclusion chromatography. The same procedure was repeated for human beta-synuclein S72C, human gamma-synuclein S92C, and mouse aSynS42C. except that they were first precipitated using the he2D Clean-Up Kit (GE Healthcare, Piscataway, NJ) as previously described (Wildburger et al., 2015, Mol Cell Proteomics 14 (5), 1288-1300) to remove excess imidazole.

### Expression of the Alpha-Synuclein1-140Ser87Cys Dimer or the Monomeric Alpha-Synuclein

All required synucleins were expressed in Rosetta™ 2 (DE3) plysS Competent Cells (Novagen). This BL21-derived E. coli strain enables enhanced expression of eukaryotic proteins through a chloramphenicol resistance (CamR)-containing plasmid that delivers the tRNAs for rare codons. Rare codons are codons that are more common in the transcription of human genes than bacterial genes and can therefore lead to problems in the transcription of proteins of human origin in other E. coli. The CamR gene allows growth in the presence of 34 μg/ml chloramphenicol (CAMP).

Transformation was performed by heat shock for 30 seconds at 42° C. To achieve a better transformation efficiency, 10 μg of the respective subcloned plasmid was added to a 20 μL aliquot of E. coli 30 min before the heat shock, after which SOC medium (Novagen) was added. After 1 h of incubation at 37° C., the E. coli were plated onto an LB agarose plate containing 34 g/mL CAMP and 0.025 mg/ml Strep to generate a selection pressure for clones containing the rare codon plasmid and the transformed plasmid. The plate was incubated overnight at 37° C. to allow colony formation. Individual colonies were selected for inoculation of a liquid LB culture with the same concentrations of CAMP and Strep and also incubated overnight. Glycerol stocks were prepared by adding 700 UL of the liquid culture to 300 μL of glycerol and stored at −80° C. The liquid culture was induced with 1 mM IPTG for 3 hours and harvested by centrifugation at 5500×g for 20 minutes. The supernatant was removed before freezing at −20° C.

### Expression of Single Domain Antibodies:

The NbSyn2 was expressed in Rosetta-Gami™ B (DE3) plysS Competent Cells (Novagen). This cell line has the advantage over the Rosetta line used for the synucleins that it additionally encodes proteins that promote the formation of disulfide bonds. The heat shock was carried out as described above. Induction with IPTG was performed at 30° C. with 0.5 mM IPTG overnight and cells were harvested by centrifugation for 20 min at 5500×g. The supernatant was removed before freezing at −20° C. Induction for NbSyn87 was performed in the same way.

### Purification of Alpha-Synuclein and Single Domain Antibodies

Lysis of E. coli cells was performed on ice by sonication after resuspension in 5 mL buffer per gram of E. coli; high salt buffer with 20 mM Imidazole. 10 μL DNAse1 and PI were used in a ratio of 1:100. Sonication was performed in 10-second pulses with 30-second pauses and the whole-cell lysate was pre-purified by centrifugation at 10,000×g for 10 minutes at room temperature in a benchtop centrifuge.

A His GraviTrap (GE Healthcare) was used to purify the His-tagged protein from the pre-purified cell lysate (PCL). After equilibrating the column with native high salt buffer containing 20 mM Imidazole, the sample was added. Washing the column with a volume of native high salt buffer containing 20 mM Imidazole removed non-ionic interactions. To remove the salt, a wash with native low salt buffer was performed. To achieve optimal elution, a Imidazole series (50, 100, 150, 200, 300, 500 mM) in low salt buffer was used. Only the fractions containing the desired protein were collected (see table below, where collected fractions are marked with an “X”).

| | 50 | | | | | |
|---|---|---|---|---|---|---|
| | mM | 100 mM | 150 mM | 200 mM | 300 mM | 500 mM |
| human | | | X | X | X | X |
| alpha- | | | | | | |
| synuclein | | | | | | |
| (αSyn hu) | | | | | | |
| αSyn hu | | | X | X | X | X |
| S87C | | | | | | |
| Nanobody | | | | | X | X |
| Syn87 | | | | | | |
| Nanobody | | | X | X | X | X |
| Syn2 | | | | | | |

To clean the column after use, 10 ml of denaturation buffer was added. After washing twice with high salt buffer containing 20 mM Imidazole, the column was stored in 20% ethanol. The secured fractions were combined in a 3 kDa Amicon (regenerated cellulose) molecular weight cutoff filter and exchanged with 20 ml of low salt buffer containing 20 mM Imidazole. The filter was stored in PBS containing 0.02% sodium azide.

Removal of Tag from Alpha-Synucleins and Single Domain Detection Antibodies

The buffer exchanged sample was filled up to 1 ml and 40 U His-tagged SUMO protease (SAE0067, Sigma/Merck) was added together with 1 mM DTT. To allow SUMO cleavage in the alpha-synucleins and single domain detection antibodies, the samples were incubated overnight at 4° C. During cleavage by the SUMO protease, the respective His-SUMO tag was cleaved off. After cleavage, the sample was re-applied to the His-GraviTrap to remove the cleaved HIS-SUMO tag and the HIS-tagged SUMO protease. The column was then re-equilibrated and the flow-through was collected with the now tagless alpha-synuclein or single domain detection antibodies.

### Biotinylation of the Purified Single Domain Detection Antibodies

For biotinylation of the single domain detection antibodies, EZ-Link™ NHS biotin (ThermoFisher) was used in a 20-fold molar excess. The biotin stock solution was prepared by dissolving 2 mg of biotin in 590 μl dimethyl sulfoxide (DMSO). The required amount of biotin was calculated according to the manufacturer's protocol and the single domain detection antibody was added. It was incubated for 30 minutes on top of each other and then buffered in a 3 kDa Amicon (regenerated cellulose) molecular weight cutoff filter with 20 ml PBS (NbSyn2) or subjected to size exclusion chromatography (SEC) on a Superdex™ 10/300 GL (GE Healthcare) (NbSyn87) to get rid of excess biotin.

Biotinylated single domain detection antibodies can be detected particularly easily using enzymes coupled to streptavidin, such as streptavidin-horseradish peroxidase.

### Method for the Detection of Dimeric Alpha-Synuclein:

His6-SUMO-NbSyn87 (oligomer assay, human) or His6-SUMO-NbSyn2 (total, pan assay) was used to coat black 384-well Nunc MaxiSorp plates (#460518, Nalge Nunc, Rochester, NY) with 20 μg/ml in a carbonate buffer (35 mM sodium bicarbonate, 16 mM sodium carbonate, 3 mM sodium azide, pH 9.6) at 20 μl/well overnight at 4° C. The plates were washed 5× between steps with PBS containing 0.005% Tween-20 in a BioTek EXL405 plate washer (BioTek, Winooski, VT). Sample plates were blocked with 0.2 μm filtered 4% Bovine Serum Albumin (BSA) (#7030, Sigma-Aldrich, St. Louis, MO) in PBS for 1 hour at room temperature. The samples or the dimer standard or alpha-synuclein monomer (for the total assay) were analyzed either neat or in standard dilution (0.2 μm filtered 0.25% BSA, 0.005% Tween-20, 3 mM sodium azide, 2 μg/mL aprotinin (EMD Chemicals, Gibbstown, NJ), 1 μg/mL leupeptin (EMD Chemicals), in PBS) was diluted to 20 μL final volume and applied to each of the His6-SUMO-NbSyn87 or His6-SUMO-NbSyn2 coated plates, respectively. Brain lysates from 9-week-old or 6-month-old mice of the Thy1 mouse model were used as samples (Chesselet et al., Neurotherapeutics. 2012 April; 9 (2): 297-314). Transgenic mice of this model express human alpha-synuclein under the control of a Thy-1 promoter. A 9-point standard curve was generated using 200, 100, 50, 25, 12.5, 6.25, 3.125, 1.56 and 0 μg/ml alpha-synuclein1-140Ser87Cys dimer or monomeric alpha-synuclein (without mutations) and loaded in triplicate in all experiments. All samples and the standard were stored on ice during processing. Plates were centrifuged at 1000×g shortly after loading samples and standards to ensure that the liquid was properly seated at the bottom of each well and to remove air bubbles. The samples and standards were equilibrated overnight at 4° C. on the plates with the single domain carrier antibodies bound to them. After a further wash step to remove unbound solutes, alpha-synuclein oligomers or total alpha-synuclein were each detected with labeled (biotinylated) NbSyn87 as a single domain detection antibody at a concentration of 100 ng/ml in PBS containing 0.2 μm filtered 0.1 mg/ml nonfat dry milk plus 0.005% Tween-20. Binding of the single domain detection antibodies to exposed alpha-synuclein oligomers or to total alpha-synuclein was allowed for 1 hour at room temperature. Poly-streptavidin HRP-20 (65R-S103PHRP, Fitzgerald, Acton, MA) was then incubated at 30 ng/ml in PBS containing 0.2 μm filtered 0.5% BSA plus 0.005% Tween-20 for 1 hour at room temperature with gentle agitation. After a final wash, the assay was developed by adding SuperSignal™ West Femto Maximum Sensitivity Substrate (34094, Thermofisher) and the luminescence was read on a Tecan Infinite M Plex plate reader. The standard curve was then used to calculate the concentration of dimeric alpha-synuclein in the samples in units of “dimer equivalents” for the oligomer assay.

FIG. 8 shows the luminescence at 650 nm of a method according to the invention for the detection of dimeric alpha-synuclein at a concentration of 0.1 μg/ml of a single domain carrier antibody His6-SUMO-NbSyn2 at different concentrations plotted on the x-axis of a single-domain detection antibody NbSyn2. At all concentrations of the single domain detection antibody, either 10 μg brain lysate (bars labeled with 20), 2 ng brain lysate (bars labeled with 22) or no sample, negative control (bars labeled with 24) were used. It can be clearly seen that at all concentrations of the single domain detection antibody the sample with 10 μg brain lysate shows the highest luminescence, while the luminescence of the samples with a low proportion of brain lysate and with no brain lysate have approximately the same luminescence. With the method according to the invention it is therefore possible to detect the dimeric human alpha-synuclein contained in the brain lysates with high sensitivity. The comparable luminescence in the samples with a low proportion of brain lysate and the negative control is partly due to non-specific binding of the single domain detection antibody to the carrier material.

### Method for the Detection of Dimeric Tau Protein:

Dimeric tau protein can be detected in the same way as described above for dimeric alpha-synuclein using the above-mentioned tau-specific antibodies. The same tau-specific antibody can be used as the single domain carrier antibody and the single domain detection antibody, so that a signal from the single domain detection antibody can only be generated if dimeric tau proteins or multimeric tau proteins are present in the sample which have two identical epitopes for binding both the single domain carrier antibody and the single domain detection antibody.

### Method for the Detection of Dimeric Beta-Amyloid:

Dimeric beta-amyloid can be determined in the same way as dimeric tau protein.

1. A method for detecting a multimeric;
peptide, comprising a first and a second peptide monomer in a sample, the method comprising:
A) providing a carrier material antibody conjugate comprising a carrier material to which a single domain carrier antibody specific for the first peptide monomer is bound,
B) contacting the sample with the carrier material antibody conjugate, wherein a complex is formed between the carrier material antibody conjugate and the multimeric peptide,
C) contacting the complex with a single domain detection antibody specific for the second peptide monomer, which comprises a detection marker, wherein the single domain detection antibody binds to the complex, wherein the single domain carrier antibody on the first peptide monomer and the single domain detection antibody on the second peptide monomer recognize the same epitope, and
D) detecting the multimeric peptide using the detection marker.

2. The method according to claim 1, wherein said carrier material antibody conjugate is used, wherein the single domain carrier antibody is bound to the carrier material via a peptide linker, wherein the peptide linker comprises a length of at least 20 amino acids and comprises an alpha-helical region.

3. The method according to claim 2, wherein the peptide linker comprises an alpha-helical region of a SUMO peptide (small ubiquitin-like modifier peptide).

4. The method according to claim 2, wherein the peptide linker is bound to the carrier material via a tag sequence.

5. The method according to claim 1, wherein the single domain carrier antibody and the single domain detection antibody are the same single domain antibody.

6. The method according to claim 1, wherein the multimeric peptide comprises or consists of a dimeric oligo- or polypeptide.

7. The method according to claim 1 for the detection of neurodegenerative diseases, wherein the multimeric peptide is at least one peptide selected from the group consisting of beta amyloid peptide dimer, tau dimer, phosphorylated tau dimer, and alpha synuclein dimer.

8. The method according to claim 7 for the detection of Parkinson's disease, wherein the multimeric peptide comprises a monomer having a sequence identity of at least 80% identical to the amino acid sequence of SEQ. ID No. 1 or tau protein or beta-amyloid protein.

9. The method according to claim 8 for the detection of Parkinson's disease, wherein the single domain carrier antibody and the single domain detection antibody are specific for alpha-synuclein.

10. The method according to claim 1, wherein in B) the single domain carrier antibody binds to the first peptide monomer, wherein an epitope recognized by the single domain detection antibody on the first peptide monomer is blocked.

11. The method according to claim 1, further comprising:
carrying out a positive control reaction, wherein as multimeric peptide for the positive control reaction a homodimer is used whose monomer has a sequence identity of at least 80% identical to the amino acid sequence of SEQ. ID No. 14, with the proviso that the amino acid cysteine at position 87 of SEQ. ID No. 14 remains unchanged.

12. The method according to claim 1, wherein in B) at least one sample selected from the group consisting of a brain- or spinal cord fluid, blood, serum, blood plasma, saliva, tissue biopsy, throat swab, nasal swab, urine, and exosome diagnostics of a patient is used.

13. The method according to claim 1, wherein the method determines whether a symptom-free patient is at risk of developing Parkinson's disease, wherein alpha-synuclein dimer is detected as a dimeric peptide.

14. A method for detecting alpha-synuclein in a sample of a patient, comprising:
A) providing a carrier material antibody conjugate comprising a carrier material to which a single domain carrier antibody specific for alpha-synuclein is bound,
B) contacting the sample comprising alpha-synuclein with the carrier material antibody conjugate, wherein a complex is formed between the carrier material antibody conjugate and the alpha-synuclein,
C) contacting the complex with a single domain detection antibody specific for alpha-synuclein which comprises a detection marker, wherein the single domain detection antibody binds to the complex, and
D) detecting the alpha-synuclein using the detection marker.

15. The method according to claim 14 for the detection of monomeric alpha-synuclein, wherein different single domain antibodies are used as single domain carrier antibodies and as single domain detection antibodies, which recognize different epitopes of alpha-synuclein.

16. The method according to claim 14 for detecting Parkinson in a human or for determining whether a symptom-free human patient is at risk of developing Parkinson's disease, wherein the single domain carrier antibody used is a single domain antibody which has a sequence identity of at least 80% identical to that of the amino acid sequence of SEQ. ID No. 11.

17. The method according to claim 14 for the detection of alpha-synuclein in vertebrates selected from | the group consisting of human, mouse, rat, gorilla, and bovine, wherein the single domain carrier antibody used is a single domain antibody having a sequence identity of at least 80% identical to that of the amino acid sequence of SEQ. ID No. 10.

18. A kit for carrying out the method according to claim 1 for detecting a dimeric peptide containing a first and a second peptide monomer in a sample, comprising:
a carrier material antibody conjugate comprising a carrier material to which a single domain carrier antibody specific for the first peptide monomer is bound,
a single domain detection antibody specific for the second peptide monomer comprising a detection marker, and
reagents for detecting the dimeric peptide using the detection marker.

19. The kit according to claim 18, further comprising:
instructions for carrying out the method for detecting a multimeric polypeptide.

20. A kit for carrying out the method according to claim 14 for detecting alpha-synuclein in a sample, comprising:
a carrier material antibody conjugate comprising a carrier material to which a single domain carrier antibody specific for alpha-synuclein is bound,
a single domain detection antibody specific for alpha-synuclein comprising a detection marker, and
reagents for detecting the alpha-synuclein using the detection marker.

21. The kit according to claim 20, further comprising:
instructions for carrying out the method according to claim 14.

22. An alpha-synuclein homodimer, wherein the homodimer comprises monomers having a sequence identity of at least 95%, identical to the amino acid sequence of SEQ. ID No. 14, with the proviso that the amino acid cysteine at position 87 of SEQ. ID No. 14 remains unchanged.

23. An expression construct, comprising:
a gene encoding an alpha-synuclein polypeptide, wherein the alpha-synuclein polypeptide has a sequence identity of at least 95% identical to the amino acid sequence of SEQ. ID No. 14, with the proviso that the amino acid cysteine at position 87 of SEQ. ID No. 14 remains unchanged.

24. An expression construct, comprising:
a gene encoding a single domain antibody specific for a peptide monomer having a peptide linker attached to the single domain antibody, wherein the peptide linker comprises a length of at least 20 amino acids and comprises an alpha-helical region.

25. The expression construct according to claim 24, wherein the peptide monomer has a sequence identity of at least 80% identical to the amino acid sequence of SEQ. ID No. 1.

26. The expression construct according to claim 24, wherein the single domain antibody has a sequence identity of at least 80% identical to that of the amino acid sequence of SEQ. ID No. 11, or wherein the single domain antibody has a sequence identity of at least 80% identical to the amino acid sequence of SEQ. ID No. 10.

27. The expression construct according to claim 24, wherein the peptide linker comprises an alpha-helical region of a SUMO peptide (small ubiquitin-like modifier peptide).

28. A carrier material antibody conjugate, comprising:
a carrier material to which a single domain carrier antibody is bound, wherein the single domain carrier antibody is bound to the carrier material via a peptide linker, wherein the peptide linker comprises a length of at least 20 amino acids and comprises an alpha-helical region.

29. The carrier material antibody conjugate according to claim 28, wherein the peptide linker comprises a sequence identity of at least 80% to the amino acid sequence of SEQ. ID No. 4, 5, 6, 7, 8 or 9.

30. The carrier material antibody conjugate according to claim 28, wherein the single domain carrier antibody has a sequence identity of at least 80% identical to the amino acid sequence of SEQ. ID No. 10 or to the amino acid sequence of SEQ. ID No. 11.

31. A single domain antibody specific for a peptide monomer having a peptide linker attached to the single domain antibody, wherein the peptide linker comprises a length of at least 20 amino acids and comprises an alpha-helical region.

32. The single domain antibody according to claim 31, wherein the single domain antibody has a sequence identity of at least 80% identical to that of the amino acid sequence of SEQ. ID No. 11, or wherein the single domain antibody has a sequence identity of at least 80% identical to the amino acid sequence of SEQ. ID No. 10.

33. The single domain antibody according to claim 31, wherein the peptide linker comprises a sequence identity of at least 80% to the amino acid sequence of SEQ. ID No. 4, 5, 6, 7, 8 or 9.
